(19) United States
(12) Patent Application Publication
(10) Pub. No.: US 2008/0021015 A1
(43) Pub. Date: Jan. 24, 2008

(54) Use as medicine of a compound restoring active principles in vivo

(75) Inventors: Jean-Robert Rapin, Paris (FR); Dominique Halbitte, Lyon (FR)

Correspondence Address: OLIFF & BERRIDGE, PLC, P.O. BOX 320850, ALEXANDRIA, VA, 22320-4850, US

(73) Assignee: DOSPHARMA, Mont St. Aignan (FR)

(21) Appl. No.: 11/879,505

(22) Filed: Jul. 18, 2007

Related U.S. Application Data
(63) Continuation of application No. 10/465,994, filed on Oct. 3, 2003, now abandoned.

(30) Foreign Application Priority Data
Dec. 29, 2000 (FR) 00.17331

(51) Int. Cl. A61K 31/4164; A61K 31/155; A61K 31/197; A61K 31/549; A61K 31/55; A61K 31/47; A61K 31/40

(52) U.S. Cl. 514/212.07; 514/223.2; 514/307; 514/399; 514/423; 514/562; 514/635; 514/789

(57) ABSTRACT

A method for delivering at least an active principal A to a human or animal, by administering, in vivo, an active compound of general formula A′ [symbol] V′ [symbol] C′, that is capable of restoring at least the entity A by cleavage, in vivo, of the corresponding attachments between A′ and V′, wherein V is a biogenic vectorization compound of general formula X-R-Y, where R represents an aliphatic, cyclic or alicyclic, saturated or unsaturated hydrocarbon chain of 2 to 10 carbon atoms, and X and Y are each a free acid, amine or alcohol function; and A and C are two respectively different active principles, one of which has a chemical function complementary to the function of X, capable of reacting with the latter to give an ionic A′ - - - V′ or covalent A′—V′ attachment that can be cleaved in vivo, and the other of which has a chemical function complementary to the function of Y, capable of reacting with the latter to give an ionic V′ - - - C′ or covalent V′—C′ attachment.

### CROSS-REFERENCE TO RELATED APPLICATION

This is a Continuation of application Ser. No. 10/465,994, filed Oct. 3, 2003, which in turn is a National Stage Application of PCT/FR01/04236, filed Dec. 31, 2001, which in turn claims the benefit of French Patent Application No. 00/17331, filed Dec. 29, 2000. The entire disclosures of the prior applications are hereby incorporated by reference.

### BACKGROUND

The invention relates to the synthesis, manufacture and use of combined medicines having preferably (but not exclusively) a complementary and/or synergistic action.

The expression “complementary action” is intended to mean the pharmacological action of two different compounds making it possible to act on the same pathology via two respectively different pharmacological mechanisms, for example the combined use of two antidiabetic agents such as a biguanide and a sulphonylurea, or making it possible to act on a main pathology and on an associated pathology, for example diabetes and a cardiovascular pathology. It is also intended to mean the pharmacological action of two different compounds, making it possible to act simultaneously, via two respectively different mechanisms, on two pathologies which are systematically associated in humans, or on a pathology and on the side effects due to the treatment of said pathology. One object of a medical combination according to the invention is to allow a bitherapy in a single dose or through the use of one and the same compound.

The expression “synergistic action” is intended to mean the pharmacological action of two compounds consisting in potentializing the potential action of at least one of said compounds, for example potentiation of the action of a biguanide by the action of a transporter, as described and proposed hereinafter, by way of example.

Conventionally, when medicines which contain two pharmaceutical active principles in combination have to be formulated, pharmaceutical formulation is used, and these combinations are, in practice, only possible when the pharmaceutical active principles chosen have comparable half-lives.

The choice of excipients which allow, despite the simultaneous administration, the sequenced or simultaneous release of the two active principles in the proportions set by the pharmacopoeia is then essential, since these excipients will condition the release of the active principles and their respective bioavailabilities.

This choice of excipients rapidly becomes extremely delicate when it is desired to formulate two active principles, since the number of physicochemical and physiological parameters to be considered is so great.

Faced with these difficulties, when two active principles must be administered simultaneously, the solution of administering active principles in two distinct pharmaceutical forms and in two distinct doses is most commonly selected.

The bioavailability and the synergy and/or complementarity of action in vivo are then dependent on the simultaneous or successive taking of the two pharmaceutical forms, and are thus difficult to predict and to measure, and, in addition, dependent on the compliance of the patient.

### BRIEF DESCRIPTION OF THE DRAWINGS

FIGS. 1-3 show exemplary formulae for arginine, metformin, and arginine hemisuccinimide-metformin hemisuccinate, respectively.

### DETAILED DESCRIPTION OF EMBODIMENTS

According to the present invention, it has been demonstrated that the use of an active compound capable of releasing in vivo, for example in the intestine, in the liver, in the plasma or other target organs, two different active principles, in sequence or simultaneously, makes it possible to resolve such problems of co-administration of different active principles, this being whatever the half-lives of the active principles used.

Thus, the present invention provides the use, as a medicine, of an active compound of general formula A′ - - - V′ - - - C′, capable of restoring at least the entity A by cleavage, in vivo, of the corresponding attachment between A′ and V′, it being specified that:
V is a biogenic vectorization compound of general formula X-R-Y, in which,
R represents an aliphatic, cyclic or alicyclic, saturated or unsaturated hydrocarbon chain of 2 to 10 carbon atoms, which is optionally substituted with C1 to C5 alkyl groups and/or with hydroxyl groups,
X and Y are each a free acid, amine or alcohol function,
A and C are two respectively different active principles, one of which comprises a chemical function complementary to the function X, capable of reacting with the latter to give an ionic A′ - - - V′ or covalent A′ - - - V′ attachment which can be cleaved in vivo, and the other of which comprises a chemical function complementary to the function Y, capable of reacting with the latter to give an ionic V′ - - - C′ or covalent V′ - - - C′ attachment.

The V′ - - - C′ or V′ - - - C′ attachment can preferably be cleared in vivo, and said active compound is also capable of restoring the entities V and C by said cleavage in vivo.

An active compound according to the present invention can, for example, be obtained by reaction, with each other, of the entities A, V and C, respectively, to give, in attached form, the radicals A′, V′ and C′, respectively.

It is understood that A and C are two respectively different active principles, for example capable of reacting in synergy, in addition to one another or in combination, i.e. one at least of these active principles, for example A, is a product, material or compound which is pharmacologically active in itself, and, for example, C is a pharmacologically active product, material or compound, or acting by potentiation of the effects of A.

This potentiation can be due to sensitization of the receptors for A, to vectorization of A with improvement of the bioavailability or to suppression of the inactivation of A.

These active principles A and C may optionally already be used in simple combination, either in the same pharmaceutical presentation or in simultaneous or combined prescriptions.

The expression “complementary chemical function” is intended to mean any chemical function capable of reacting with a free or terminal function of the biogenic compound. For example, V has to comprise a function reacting with A and a function reacting with C. Thus, if A and C each have an acid function, V is a diamine, a dialcohol or an alcohol-amine, so as to form an amide, an ester or a salt, respectively. Thus, if A and C each have an amine function, V is a diacid so as to form an amide or a salt. If A and C each have an alcohol function, V is a diacid so as to form a diester. According to the principle of the invention, all compositions are possible. Consequently, if A has an acid function and C an alcohol function, V is, for example, an alcohol-amine in order to act with the acid function of A to give an amide, an ester or a salt, and with the alcohol function of C to give an ester.

The expression “covalent attachments” is herein intended to mean chemical attachments capable of being formed by the reaction of so-called complementary chemical functions, between the biogenic vectorization compound V and the active principles A and C.

The expression “ionic attachments” is herein intended to mean attachments by electrostatic force, capable of being formed by action of the so-called complementary chemical functions, between the biogenic vectorization compound V and the active principle A or C, therefore attachments of the acid salt, amine salt, alkoxide and acid/base type, this taking place independently from the molar proportion existing between the compound V and the active principle A or C, belonging to the complex formed by said ionic attachments.

The expression “attachment which can be cleaved in vivo” is intended to mean any attachment allowing the release and restoration of the active principles A and C and of the biogenic vectorization compound V, in vivo, by breaking of the ionic or covalent attachments between the complementary chemical functions of A and V, and of C and V.

The covalent attachments which can be cleaved are cleaved by action of the enzymes present in the in vivo medium of the site of release. Since the covalent attachments are amide attachments or ester attachments, the enzymes involved in this cleavage are amidases, esterases and hydrolases. These enzymes are present, in particular, in the digestive tract (oral administration), predominantly in the liver and in the blood, and are potentially present in the target organs.

Amidases which hydrolyse the attachment —CO—NH— are found in the liver, they are relatively inactive; hence an expected sustained effect with the compound according to the invention bearing such an attachment. Some of these amidases are known; they are endopeptidases which hydrolyse gamma-amine-containing or gamma acid attachments. According to the invention, V can in fact be a gamma-amino acid with a second acid or amine function in the gamma position (in the case of glutamic acid or of lysine, for example).

Esterases which hydrolyse the attachment —CO—O— are extremely numerous in living organisms. They are, however, ubiquitous and relatively nonspecific for a substrate, hence a high reaction rate, with rapid release of the constituents A, V, C of the active compound according to the present invention. Those most specific for a substrate bear the name of the substrate and, by way of this, mention may be made, for example, of cholinesterases or procaine esterases.

Hydrolases also hydrolyse esters and all large molecules supplied to the organism in the form of foods. These hydrolases are numerous and ubiquitous also. They are, however, specific for the biogenic vectorization compound V used.

As cleavage enzymes which can be used for implementing the present invention, mention may be made of proteolytic enzymes such as pepsin, trypsin, catalases and endo- and exopeptidases. Enzymes which can also be used are amylases and osidases and, finally, lipases and beta-oxygenases for the destruction of lipids.

These enzymes are only involved when the structure of the biogenic vectorization compound comprises one or more attachments which are capable of being cleaved. For example, the lipase acts if the biogenic vectorization compound is a long chain diacid (8 to 10 carbon atoms, comparing it to a fatty acid), and the attachment A—V or V—C is obtained by condensation with a secondary alcohol function of A or C.

The ionic attachments which can be cleaved are cleaved as a function of their site of release, for example intestine, liver, plasma or target organ, it being understood that acid salts, amine salts or alkoxides are generally ionized at the pHs of the media of living organisms. Generally, the pH is between 2 and 8, and is, for example, 2 for the stomach and 6 for the intestine, for example.

There is, therefore, ionization of the active compound according to the invention, as a function of the type of salt used, and dissociation of said active compound, when the latter comprises at least one ionic attachment. The salt is chosen as a function of its dissociation constant and of the pH of the in vivo site of release. For example, for dissociation in the stomach, a salt of a weak acid and of a strong base is chosen.

The choice of the biogenic vectorization compound, and in particular choice of its free functions X and Y, is made according to the nature of the free and complementary chemical functions present in or on the active principles A and C intended to be vectorized, i.e. attached by covalent or ionic attachment to this biogenic vectorization compound, but also according to the chosen sites of cleavage and release. This biogenic vectorization compound is a product which is of natural or unnatural origin and/or is metabolizable and/or is biodegradable and/or is atoxic with respect to humans or to animals, at a physiological dose. This biogenic vectorization compound will be chosen from biologically tested and described compounds, for example gamma-amino acids involved in protein synthesis, diacids involved in the Krebs cycle and ethanolamines which constitute cell membranes, which are metabolizable and atoxic, and capable of being integrated, themselves or their metabolites, into the major biological cycles of life. By way of a biogenic vectorization compound, mention may be made, for example, of the succinic acid which is found in the Krebs cycle or methyl succinic acid which is biodegraded to succinic acid.

Any active principle is a chemical, biochemical or biological molecule which is natural or obtained by human hand, for example by synthesis or via the recombinant pathway. This molecule has a demonstrated biological activity for treating or preventing any organic or functional disorder or disease in humans or animals. This activity has, for example, an effect which is proportional to the dose, or a dualism of action, this biological activity being objectively demonstrated or demonstratable. They are, in particular, pharmacologically and therapeutically active substances already known per se or still to come.

The different active principles A and C, capable of acting in synergy and/or in addition to each other, are preferably chosen from active principles which have approximately equal half-lives, belong to the same therapeutic class and act on the same pathology via two different mechanisms of action, or which belong to different therapeutic classes and make it possible to treat systematically associated polypathologies, for example a main pathology treated with a first active principle and a secondary pathology treated with a second active principle, said secondary pathology being caused by the administration of the first active principle.

The pharmacological actions of the active principles selected are therefore, for example, either complementary or synergistic. If the actions are synergistic or if there is potentiation, for example, the decrease in the doses will enable the side effects to be decreased.

These active principles will be chosen, in particular:
as a function of their capacity for acting in synergy and/or in addition to each other,
as a function of their capability for attaching to a biogenic vectorization compound, and
as a function of their biochemical or metabolic capacity for being released in vivo, by cleavage of the attachments attaching them to the biogenic vectorization compound selected, by enzymatic action or as a function of the in vivo pH at the site of release.

The attachments selected between the biogenic vectorization compound and the active principles A and B depend on the possible metabolisms at the gastrointestinal and hepatic level.

For example, the salts can be dissociated in the digestive tract, the hydrolysis possibly being delayed using gastro-resistant pharmaceutical forms. The esters are hydrolysed in acid medium, or hydrolysed by the esterases of the gastric juices, the hydrolysis also possibly being delayed using gastro-resistant pharmaceutical forms. The amides are hydrolysed by the hepatic amidases, the kinetics of these hydrolases being generally slow.

Thus, in order to achieve an active compound AVC which can be used as a medicine according to the invention, the following steps are necessary:
choice of the active principles as a function of the target(s) which is (are) the therapeutic object(s), and of the presence or absence on this active principle of free and accessible chemical functions capable of being chemical functions complementary to those of the biogenic vectorization compound, i.e. the presence, for example, on this active principle of acid, amine or alcohol functions which are reactive.
choice of the biogenic vectorization compound as a function of the complementary chemical functions of the active principles selected A and C and of the qualities of the biogenic vectorization compound: said biogenic compound selected is metabolizable and/or biodegradable and/or atoxic with respect to humans or to animals, at a physiological dose. It is chosen from described or established compounds which are biologically easily absorbed.
validation of the possibility of synthesizing the potential compounds AVC and,
choice of the final active compounds from among the potential compounds, by sorting as a function of the results of the assays of cleavage as a function of the targeted sites of release, and then as a function of the results of the toxicity assays.

The acid, amine or alcohol functions which are suitable for the implementation of the invention are those whose reactivity is not hindered by problems of steric hindrance, for example, or by the proximity of substituents which modify the electro-activity of these chemical functions.

The synthetic pathways selected are, for example, those generally used for the formation of double salts, diesters, diamides, ester salts, amide salts or ester amides, i.e. general methods of synthesis with protection/deprotection as a function of the chemical functions present and of their respective reactivities.

Thus, for example, with a biogenic vectorization compound comprising two acid functions, one of the caboxyls is protected with a methyl group, the other being in very reactive form, for example in the form of acid chloride, so as to react with the first active principle, for example A, the protected function then possibly being released by gentle hydrolysis in order to be able to react with the second active principle, for example C.

The sequence of the reactions is then preferably, for example, as follows:
synthesis of the amide of the biogenic vectorization compound by formation, for example, of an acid chloride or of an anhydride, and then reaction with the amine function of the active principle A, the other acid function being, for example, protected by formation of an ester.
after formation of the amide, the other acid function of said biogenic compound is deprotected by hydrolysis of the ester, and the formation of an amine salt or of an ester salt, with the active principle C, is again possible.

For example, a compound of formula A′ [symbol] V′ [symbol] C′ is thus obtained, in which the attachment between A′ and V′ is produced by the formation of an amide attachment, and in which the attachment between V′ and C′ is obtained by the formation of a salt between an amine and an acid.

Various assays can be carried out in order to evaluate the ability of the attachments A′ [symbol] V′ and V′ [symbol] C′ to be cleaved in vivo and of the active principles A and C to be correspondingly released. These assays consist, for example, in observing the release of the active principles in an intestinal juice or studying the hepatic metabolism on rat hepatocyte primary cultures. These two assays are described hereinafter.

In Vitro Assay of Cleavage in an Intestinal Juice

A preparation of intestinal juice containing trypsin, peptidases, lipase, amylase and all the other enzymes of the exocrine pancreas is used. This assay is validated beforehand using calibration compounds. A known amount (of the order of one microgram) of the compound A′V′C′ is mixed together with a known amount of intestinal juice (the trypsin and lipase contents of which are controlled). The reaction mixture is kept at 37° C. for one hour. This time is compatible with the intestinal transit. Samples are taken every 15 min, and the products A and C are detected and their concentration measured using HPLC coupled to a UV detector, or a mass spectrometer if it is not possible to use UV light. The columns used depend on the nature of A and of C, but are generally ion exchange columns, because of the presence of released alcohol, amine or acid forms. After calibration, the total amount of A or of C released in one hour determined, and the intermediate points, making it possible to calculate the dissociation constants Km and the rate Vmax of the enzymes for the active compound A′V′C′ used. This assay can be coupled with determination of the release of A, C and V in the gastric juice, using exactly the same principle, but replacing the intestinal juice with gastric juice.

In Vitro Assay on Rat Hepatocyte Primary Cultures

A primary culture of rat hepatocytes, which are close to those of humans for metabolism studies, in a HEPES medium is used, to which a known amount of compound A′V′C′ of the order of one microgram is added. The products are left in contact for 6 hours and samples, on which the supernatant is isolated and the hepatocytes in the pellet are lysed, are taken at 1 hour, 2 hours and 4 hours. In these media, the concentrations of released active principles A and C are measured. As above, it is possible to calculate the Vmax and Km of the enzymes involved in the metabolism.

When the compounds according to the invention do not cross the cell membranes, the same type of study can be carried out on a rat liver homogenate.

The possible toxicity of the biogenic vectorization compound, V is related to that of the active compound A′V′C′ according to the invention. As this active compound is metabolized to A, C and V, and V is a substance which is by definition biological, the toxicity of the compound according to the invention must be compared to the sum of the toxicities due to the administration of the active principle A and of the active principle C. In addition, when the active compound combines two active principles having, under these conditions, at least for one said active principle, an efficacy greater than that of said same active principle alone, said compound can be considered to be less toxic. However, a method for predicting the toxicity, alternative to the standard methods in vivo, is proposed hereinafter for comparing the toxicity of A and C and of A′ [symbol] V′ [symbol] C′ at identical concentrations expressed as A or as C (see Toxicologic Emergencies, Sixth Edition 1997, Goldfranck et al. Appleton and Lange, Connecticut, USA).

In Vitro Toxicity Assay

A method for culturing primary hepatocytes over a period of 96 hours is used (see Biochemical Pharmacology, vol. 50, 1995, pp. 775-780). The hepatocytes are isolated in situ by collagen profusion. They are then placed in a Williams medium supplemented with foetal calf serum, with cortisol and with glutamine, in a proportion of 1 million cells per well. Increasing and toxic concentrations of A and C, and of A′ [symbol] V′ [symbol] C′ are then added to each well. Samples are taken up after 6 h, 12 h, 24 h, 48 h and 96 h and the viability of the cells is determined with a methylene blue test, by albumin expression, by hepatocyte apoptosis and by measuring cytochrome P450 activity.

The viability of the cells with the methylene blue test gives results similar to those obtained with an LD 50.

The results obtained by albumin expression make it possible to learn the limits of tolerance of the hepatocyte to any toxic substance (end toxicity). Specifically, one of the main roles of the hepatocyte is to synthesize proteins. During a toxic effect, this expression of the synthesis and release of albumin is modified.

The results obtained by hepatocyte apoptosis make it possible to confirm the end toxicity since, during contact with a toxic substance, the cells will programme their destruction, which corresponds to the phenomenon of apoptosis which is measured by the abnormal DNA.

The measurement of the cytochrome P 450 activity documents the phenomena of induction and of inhibition of these enzymes, often encountered with pharmacologically active products. A series of assays makes it possible to determine the activity of the P 450 cytochrome isoforms.

The synthesis of active compounds of general formula A′ [symbol] V′ [symbol] C′ according to the invention, which allow, by cleavage in vivo, the simultaneous administration of two active principles A and C with complementary action and with antibiotic action, is performed by reacting, with a biogenic vectorization compound V, a sulphamide such as sulphamethoxazole and trimethoprim, for example.

The synthesis of active compounds of general formula A′ [symbol] V′ [symbol] C′ according to the invention, which allow, by cleavage in vivo, the simultaneous administration of two active principles A and C with complementary action and with anti-ulcer action, is performed by reacting, with a biogenic vectorization compound V, ranitidine and azole, for example.

The synthesis of active compounds of general formula A′ [symbol] V′ [symbol] C′ according to the invention, which allow, by cleavage in vivo, the simultaneous administration of two active principles A and C with complementary action and with anti-rheumatism action, for the treatment of arthritis, is performed by reacting, with a biogenic vectorization compound V, for example a nonsteroidal anti-inflammatory agent and penicillamine.

The synthesis of active compounds of general formula A′ [symbol] V′ [symbol] C′ according to the invention, which allow, by cleavage in vivo, the simultaneous administration of two active principles A and C with synergistic action, one of which is an antidiabetic agent, is performed by reacting, with the biogenic vectorization compound, for example metformin and arginine, which by virtue of its transporter role allows the potentiation of the action of the metformin.

The synthesis of active compounds of general formula A′ [symbol] V′ [symbol] C′ according to the invention, which allow, by cleavage in vivo, the simultaneous administration of two active principles with combined action, is performed by reacting, with the biogenic vectorization compound, for example an anti-hypertensive agent such as a conversion enzyme inhibitor, for example quinapril, benazepril and captopril, and a diuretic such as hydrochlorothiazide, in the treatment of hypertension, or, for example an anti-ulcer agent such as ranitidine, and an antibiotic such as metronidazole, in the treatment of gastrointestinal ulcer with helicobacter infection.

The synthesis of active compounds of general formula A′ [symbol] V′ [symbol] C′ according to the invention, which allow, by cleavage in vivo, the simultaneous administration of two active principles with complementary action, by action on the side effects systematically associated with a therapeutic treatment, is performed by reacting, with a biogenic vectorization compound, a nonsteroidal anti-inflammatory agent such as diclofenac or naproxen, and an anti-ulcer agent such as cimetidine, for example.

By way of example, an active compound which can be used as a medicine in the treatment of diabetes and which is capable of restoring, by cleavage in vivo, metformin (first active principle) and arginine (second active principle), is prepared using succinic acid as the biogenic vectorization compound in order to synthesize arginine hemisuccinimide-metformin hemisuccinate.

The method for preparing this active compound, given by way of example, comprises the following steps:
reaction of the succinic acid monochloride monoester, in solution in ether or in benzene, with the arginine in aqueous solution in sodium carbonate,
release of the metformin base from the hydrochloride in concentrated sodium hydroxide medium, and extraction with absolute alcohol,
formation of the salt of arginine hemisuccinimide with metformin.

The present invention is now described by way of example with reference to two active principles, that is to say, metformin (active principle A), and arginine (active principle C), attached to a biogenic compound V consisting of succinic acid; the latter reacts, on the one hand, covalently with an amine function of arginine and, on the other hand, ionically (salification reaction) with an amine function of metformin.

Synthesis of the Arginine Hemisuccinimide-Metformin Hemisuccinimate

a) First Step: Preparation of the Arginine Hemisuccinimide.

Arginine base (6 g) is dissolved in 120 ml of an aqueous solution of sodium carbonate (N=10.6 g/100 ml). Moreover, succinic monochloride monoester is diluted in 50 ml of sulphuric ether, with a slight excess of succinic monochloride monoester for a reaction which is mole for mole with respect to the arginine. The ether-containing solution is added to the aqueous solution in 10 minutes, with vigorous stirring at room temperature. The reaction liquid is maintained for one hour with vigorous stirring, while heating slowly for complete distillation of the ether. The mixture is evaporated to dryness, and the residue is taken up with a minimum volume of distilled water (20 ml) and acidified with diluted hydrochloric acid. By concentrating (gentle heating under partial vacuum), white crystals of arginine hemisuccinimide are obtained.

The NMR spectrum, the elemental analysis and the purity of the product by thin layer chromatography are verified. In particular, the presence of the arginine amino acid residue is verified by the ninhydrin reaction, and the presence of the free carboxyl of the succinic acid is verified by titrimetry.

The yield is quantitative.

b) Second Step: Release of the Metformin Base.

Ten grams of metformin hydrochloride are added to 40 ml of a 5N sodium hydroxide solution. The reaction mixture is heated for two hours at 40° C. After evaporation under vacuum at 40° C., the viscous residue is taken up with 100 ml of absolute ethanol. Filtration makes it possible to eliminate the impurities, and an insoluble residue of sodium chloride remains. The metformin base is in alcoholic solution, and it is isolated in the form of a viscous powder by evaporation. The NMR spectrum confirms the structure of the metformin. The absence of chloride is verified with silver nitrate.

It is recalled that metformin, i.e. N,N-dimethylimidodicarbonimidic diamide, is identified in the MERCK Index under the number 5792, and characterized under the Chemical Abstracts number 657—24-9.

c) Third Step:

The metformin base is added, mole for mole, to an aqueous solution of arginine hemisuccinimide. It dissolves immediately.

The water is completely evaporated off at 60° C. under vacuum. The residue is redissolved in distilled water, and crystallizes during concentration under vacuum.

Translucent crystals are obtained, which are soluble in water and insoluble in organic solvents. The melting point is 188-189° C.

The NMR spectrum, the elemental analysis and the presence of a single spot after thin layer chromatography confirm the structure and the purity of the product. The total yield is quantitative.

After the above reactions, the yield is close to 90%. The losses originate from the purifications and filtrations.

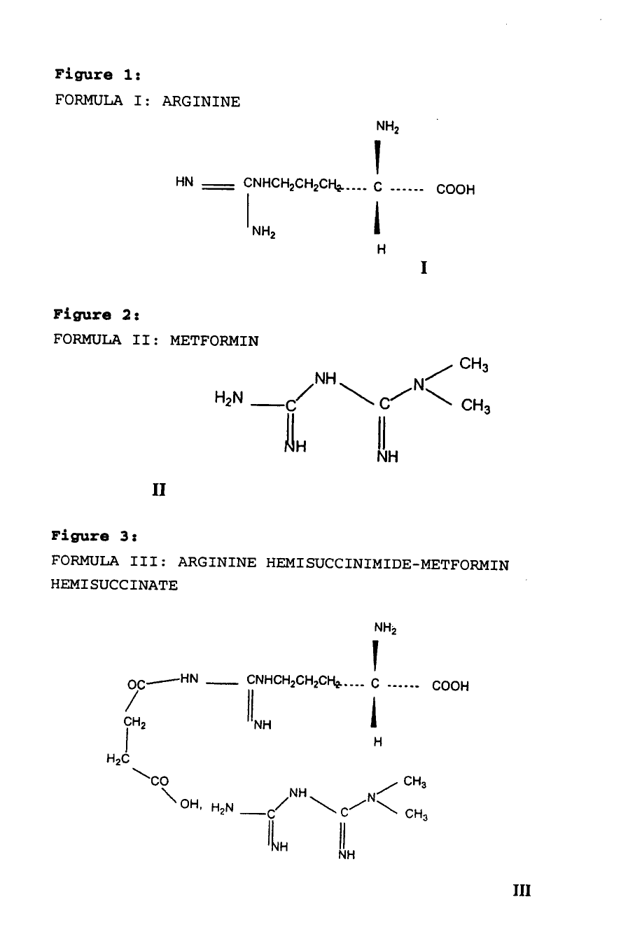

The developed formulae of the arginine, of the metformin and of the salt of arginine hemisuccinimide with metformin are given in FIGS. 1 to 3 respectively.

Cleavage Assay:

This assay is carried out according to the in vitro method in an intestinal juice, described above, according to the in vitro toxicity assay described. Immediate release of the metformin without modifying the arginine hemisuccinimide part is observed. A second assay is carried out on a rat hepatocyte culture, according to the method described above. Slow release of arginine over 24 hours is observed.

Toxicity:

This assay is carried out according to the in vitro toxicity assay described above. The toxic dose is observed with the metformin at 10⁻² M, and it is identical for the active compound A′-V′-B′, namely the salt of arginine hemisuccinimide with metformin.

Verification of the Pharmacological Activity of the Active Compound Obtained

The kinetic and pharmacological advantage of the active compound according to the present invention is described hereinafter, taking, as an illustrative example, the arginine hemisuccinimide-metformin hemisuccinate and a metformin hydrochloride/arginine hydrochloride combination:
a) A pharmacokinetic study carried out in two groups of 20 rats each, receiving, orally, 50 mg/kg of metformin hydrochloride and 50 mg/kg of arginine hemisuccinimide-metformin hemisuccinate, respectively, made, it possible to calculate the various kinetic parameters. The arginine hemisuccinimide-metformin hemisuccinate releases metformin and, in the two groups, it is the plasmatic levels of the metformin which are determined.

After administration of 50 mg/kg of metformin hydrochloride, the concentration peak is observed within 90 minutes and is found to be 3.9 μg/ml. The bioavailable fraction is 60% and the half-life is on average 2.5 hours.

The administration of 50 mg/kg of arginine hemisuccinimide-metformin hemisuccinate corresponds to approximately 25 mg/kg of metformin hydrochloride, namely a half dose. The concentration peak is observed at 60 minutes and it is found to be 2.9 μg/ml of metformin. The bioavailable fraction is 75% and the half-life is 2.6 hours.

These results demonstrate that the entry of the metformin (total amount and rate of transfer) is improved in the case of the arginine hemisuccinimide-metformin hemisuccinate.

The antidiabetic activity was studied, from the pharmacological point of view, on two models of rats made diabetic.

The first model consisted in treating the rats with streptozotocin (50 mg/kg, IP), this being a compound which induces an increase in glycaemia, which increases from 5.5 mM to 12-14 mM in 21 days. The administration of metformin (30 mg/kg) significantly decreases this hyperglycaemia, which decreases from 12.11 to 9.85 mM on average. At the same dose of 30 mg/kg (approximately two times less metformin base), the arginine hemisuccinimide-metformin hemisuccinate decreases more considerably the hyperglycaemia, which drops from 12.66 to 7.56 mM. The difference between the two treatments is significant despite the lower dose of metformin.

The second study is carried out with the administration of fructose at 10% in the drinking water of the rats. An insulin resistance develops, followed by diabetes of non-insulin-resistant type. The arginine hemisuccinimide-metformin hemisuccinate proves to be significantly more active than the metformin alone, at an equivalent dose of metformin base.

A study on the hamster cheek pouch shows that the arginine hemisuccinimide-metformin hemisuccinate reproduces at least the effects of the two active principles on the microcirculation, namely the vasodilatory action of the arginine and the action of the metformin on vasomotion.

What is claimed is:

1. A method for delivering at least an active principal A to a human or animal, comprising administering, in vivo, an active compound of general formula A′ [symbol] V′ [symbol] C′, capable of restoring at least the entity A by cleavage, in vivo, of the corresponding attachments between A′ and V′, wherein:
V is a biogenic vectorization compound of general formula X-R-Y, where:
R represents an aliphatic, cyclic or alicyclic, saturated or unsaturated hydrocarbon chain of 2 to 10 carbon atoms, which is optionally substituted with C1 to C5 alkyl groups and/or with hydroxyl groups, and
X and Y are each a free acid, amine or alcohol function, and
A and C are two respectively different active principles, one of which comprises a chemical function complementary to the function X, capable of reacting with the latter to give an ionic A′ - - - V′ or covalent A′—V′ attachment which can be cleaved in vivo, and the other of which comprises a chemical function complementary to the function Y, capable of reacting with the latter to give an ionic V′ - - - C′ or covalent V′—C′ attachment.

2. The method according to claim 1, wherein the V′ - - - C′ or V′—C′ attachment can be cleaved in vivo, and said active compound is also capable of restoring the entities V′ and C by said cleavage in vivo.

3. The method according to claim 1, wherein the functions of X and Y are respectively different.

4. The method according to claim 1, wherein the functions of X and Y are identical.

5. The method according to claim 3, wherein the function of X is an acid or amine function, and the function of Y is an alcohol function.

6. The method according to claim 4, wherein the functions of X and Y are each an acid function.

7. The method according to claim 6, wherein the attachment A′—V′ is covalent and of the amide type.

8. The method according to claim 6, wherein the attachment V′ - - - C′ is ionic and of the salt type.

9. The method according to claim 1, wherein the active compound has as a general formula A′—V′—C′, the attachments A′—V′ and V′—C′ each being of the amide or ester type.

10. The method according to claim 1, wherein the active compound has as a general formula A′ - - - V′ - - - C′, the attachments A′ - - - V′ and V′ - - - C′ being of the ionic type and respectively different, and each being an attachment of the salt or acid/base type.

11. The method according to claim 1, wherein the active compound has as a general formula A′ - - - V′—C′, the attachment A′ - - - V′ being of the ionic type and the attachment V′—C′ being of the covalent type.

12. The method according to claim 1, wherein the active principles A and C have an approximately equal plasmatic half-life.

13. The method according to claim 1, wherein the active principles A and C belong to the same therapeutic class or respectively different therapeutic classes.

14. The method according to claim 1, wherein the active principles make it possible to treat two systematically and respectively associated pathologies.

15. The method according to claim 1, wherein the biogenic vectorization compound is metabolizable and/or biodegradable and/or atoxic in human or animals.

16. A process to achieve an active compound AVC for delivering at least one active principle A to a target human or animal which can be used as a medicine, comprising:
a) choosing an active principle A as a function of at least one target which is a therapeutic object, and of the presence or absence on this active principle of free and accessible chemical functions capable of being chemical functions complementary to those of a biogenic vectorization compound;
b) choosing the biogenic vectorization compound as a function of the complementary chemical functions of the selected active principles A and C and of the qualities of the biogenic vectorization compound, said biogenic vectorization compound being at least one of metabolizable, biodegradable, and atoxic with respect to humans or to animals, at a physiological dose;
c) validating the possibility of synthesizing potential compounds AVC; and
d) choosing a final active compound from among the potential compounds, by sorting as a function of the results of the assays of cleavage as a function of the targeted sites of release, and then as a function of the results of toxicity assays.

17. The process according to claim 16, wherein at step c, the synthetic methods selected are those used for the formation of double salts, diesters, diamides, ester salts, amide salts or ester amides.

18. The process according to claim 16, wherein the biogenic vectorization compound is a compound of general formula X-R-Y, wherein:
R represents an aliphatic, cyclic or alicyclic, saturated or unsaturated hydrocarbon chain of 2 to 10 carbon atoms, which is optionally substituted with C1 to C5 alkyl groups and/or with hydroxyl groups, and
X and Y are each a free acid, amine, or alcohol function.

19. The process according to claim 16, wherein the active principles chosen at step a) are a sulphamide chosen among sulphamethoxazole and a trimethoprim.

20. The process according to claim 16, wherein the active principles chosen at step a) are selected from the group consisting of:
ranitidine and metronidazole;
a nonsteroidal anti-inflammatory agent and penicillamine;
metformin and arginine; and
an anti-hypertensive agent chosen among quinapril, benazepril and captopril, and a diuretic chosen among hydrochlorothiazide.
